(19) United States
(12) Patent Application Publication
(10) Pub. No.: US 2012/0054878 A1
(43) Pub. Date: Mar. 1, 2012

(54) METHOD AND SYSTEM FOR ANALYZING OPTICAL SIGNAL

(75) Inventor: Hirobumi Suzuki, Hino-Shi (JP)

(73) Assignee: OLYMPUS CORPORATION, Tokyo (JP)

(21) Appl. No.: 13/288,474

(22) Filed: Nov. 3, 2011

Related U.S. Application Data
(62) Division of application No. 12/724,795, filed on Mar. 16, 2010.

(30) Foreign Application Priority Data
Mar. 24, 2009 (JP) 2009-071577

(51) Int. Cl. C12Q 1/66; C12Q 1/02; G01N 33/50

(52) U.S. Cl. 800/3; 435/8; 435/29

(57) ABSTRACT

The present invention is related to a method of analyzing an optical signal which analyzes a signal substance induced by a photosensitive protein, includes the steps of introducing a gene which expresses a luminescent probe to analyze the signal substance into an organism sample, emitting a stimulus light to activate the photosensitive protein, and detecting an optical signal emitted by the organism sample.

### CROSS-REFERENCE TO RELATED APPLICATIONS

This application is a divisional of U.S. patent application Ser. No. 12/724,795, filed on Mar. 16, 2010, which is based upon and claims the benefit of priority from prior Japanese Patent Application No. 2009-071577, filed Mar. 24, 2009, the entire contents of each of which are incorporated herein by reference.

### BACKGROUND OF THE INVENTION

1. Field of the Invention

The present invention relates to a method of analyzing an optical signal which analyzes a signal substance, for example, a signal protein induced by a photosensitive protein.

2. Description of the Related Art

A signal protein is a protein group constituting a series of reaction systems initiated by activation of a specific protein (hereinafter, also referred to as an initiator protein) such as a receptor. A gene that determines a phenotype (hereinafter, also referred to as a phenotypic determination gene) such as a pathological gene is expressed in the end after passing through a reaction cascade by the signal protein. Analysis of the signal protein is indispensable for development of a new drug that enables expression control of a pathological gene so that the analysis is widely conducted at development sites of a new drug.

Various initiator proteins that activate a signal protein are known. A channel protein present in a cell membrane has been attracting particular attention because the channel protein can become a trigger of a reaction cascade by an intricate and important signal protein. As one of the channel proteins, there is a photosensitive channel protein that causes depolarization and hyperpolarization of a channel (that is, channel opening/closing) by photic stimulation of a specific wavelength.

For example, channel rhodopsin-2 (ChR2) and halorhodopsin (NpHR) are photosensitive ion channel proteins derived from green algae. Opening/closing of a channel of sodium ions or chlorine ions of nerve cells can be controlled by causing nerve cells of mammals to express ChR2 and NpHR and providing photic stimulation of a specific wavelength. A response of opening/closing of an ion channel by photic stimulation is detected electrophysiologically through a signal from electrodes as a change in action potential of nerve cells (Boyden et al, Nature Neuroscience, 8: 1263-1268 (2005)).

For detection on an individual level, transgenic mice constantly expressing genes of these photosensitive ion channel proteins are engineered and a surgical operation is carried out on them so that only a specific region such as the center of motion can be irradiated with light through an optical fiber, whereby changes in behavior of mice can be observed with photic stimulation (Zhang et al, Nature Reviews, Neuroscience, 8: 577-581 (2007) and Gradinaru et al, J. Neuroscience, 27: 14231-14238 (2007)).

The action potential of nerve cells described above is caused by opening/closing of a channel, which is an initiator protein. Changes in behavior of mice are caused by the expression of a phenotypic determination gene. However, a new label becomes necessary to analyze the expression of a signal protein linking the initiator protein and phenotypic determination gene.

Methods of monitoring such a signal protein include an observation technique by a fluorescence microscope using a fluorescent probe. By using, for example, a fluorescent probe whose ratio of fluorescence intensity or fluorescence wavelength changes when bound to calcium ions, which are a typical signal protein, the calcium ions can be monitored. Similarly, by using a fluorescent probe obtained by fusing a transcription factor, which is a typical signal protein, with a green fluorescence protein (GFP), the transcription factor can be monitored. For observation under a fluorescence microscope using a fluorescent probe, a fluorescent substance is irradiated with excitation light of a specific wavelength and changes in fluorescence intensity of a specific fluorescence wavelength emitted from the fluorescent substance are monitored.

### BRIEF SUMMARY OF THE INVENTION

When, for example, changes in concentration of calcium ions, which are a signal transmitter, positioned downstream from ChR2 or NpHR described above are monitored, ChR2, which is an ion channel of sodium, responds to photic stimulation of blue near 470 nm and NpHR, which is an ion channel of chlorine, responds to photic stimulation of orange near 580 nm. However, Fluo3 frequently used as a calcium probe is excited by a blue light near 480 nm and emits a green light near 500 nm. Thus, the wavelength band (470 nm) of photic stimulation of ChR2 and the wavelength band (480 nm) of excitation light of a fluorescent probe interfere with each other. That is, excitation light of a fluorescent probe simultaneously acts also for photic stimulation of a channel protein (initiator protein) so that photic stimulation of the channel protein becomes excessive, making a correct signal analysis impossible.

Moreover, when monitoring a transcription factor, while fluorescent probes of various excitation wavelengths caused to bind to the transcription factor exist, types of usable fluorescent probes are extremely limited because the wavelength band used for photic stimulation of a channel protein cannot be used and thus, for example, fluorescent probes such as DsRed that cause excitation in the wavelength band of orange cannot be used.

Therefore, an object of the present invention is to provide a method of analyzing an optical signal capable of monitoring a signal substance easily and correctly by settling the issue of interference caused between a stimulus light to activate a photosensitive protein and an excitation light of a fluorescent probe of the signal protein.

As a result of intensive research, the inventors of the present invention came to settle the above issue by using a luminescent probe to monitor a signal substance, instead of a fluorescent probe. The term “a signal substance” used herein means a substance associated with a signaling in an organism, for example, a signal protein and a signal transmitter. Hereinafter, the description is going on with a using the term “a signal protein”. However, unless a specific statement, the term “a signal protein” may be read into the words “a signal transmitter”.

Now, one aspect of the present invention provides a method of analyzing an optical signal which analyzes a signal protein induced by a photosensitive protein, comprising the steps of:

introducing a gene which expresses a luminescent probe to analyze the signal protein into an organism sample;

emitting a stimulus light to activate the photosensitive protein; and

detecting an optical signal emitted by the organism sample.

Another aspect of the present invention provides a method of analyzing an optical signal which analyzes first and second signal proteins induced by a photosensitive protein, comprising the steps of:

introducing a gene which expresses a first luminescent probe to analyze the first signal protein and a gene which expresses a second luminescent probe to analyze the second signal protein into an organism sample;

emitting a stimulus light to activate the photosensitive protein; and

detecting, among the optical signals emitted by the organism sample, an optical signal derived from the first signal protein and an optical signal derived from the second signal protein.

Another aspect of the present invention provides an optical signal analysis system which analyzes a signal protein induced by a photosensitive protein, comprising:

a stimulus light emitting unit which emits a stimulus light to activate the photosensitive protein; and

a luminescent image pickup unit which picks up a luminescent image in which an optical signal emitted by an organism sample is formed.

Another aspect of the present invention provides an optical signal analysis system which analyzes first and second signal proteins induced by a photosensitive protein, comprising:

a stimulus light emitting unit which emits a stimulus light to activate the photosensitive protein; and

a luminescent image pickup unit which picks up a luminescent image in which, among optical signals emitted by an organism sample, an optical signal derived from the first signal protein and an optical signal derived from the second signal protein are separately formed.

According to the method of analyzing an optical signal in the present invention, the issue of interference caused between a stimulus light to activate a photosensitive protein and an excitation light of a fluorescent probe of a signal protein can be settled so that the signal protein can be monitored easily and correctly.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a flow chart of a method of analyzing an optical signal in a first embodiment;

FIG. 2 is a schematic diagram of an optical signal analysis system 100 used in the first embodiment;

FIG. 3 is a first schematic diagram of a luminescent image pickup unit 120 in an optical signal analysis system used in a second embodiment; and

FIG. 4 is a second schematic diagram of the luminescent image pickup unit 120 in the optical signal analysis system used in the second embodiment.

### DETAILED DESCRIPTION OF THE INVENTION

Embodiments of the present invention will be described below using the drawings. Each embodiment shown below is only an exemplary embodiment to describe the configuration of the present invention in detail. Therefore, the present invention should not be specifically interpreted based on the description of each embodiment below. The scope of the present invention includes all embodiments including various modifications of each embodiment below and improvements thereof without departing from the scope of the general inventive concept as defined by the appended claims and their equivalents.

1. Method of analyzing an optical signal

### First Embodiment

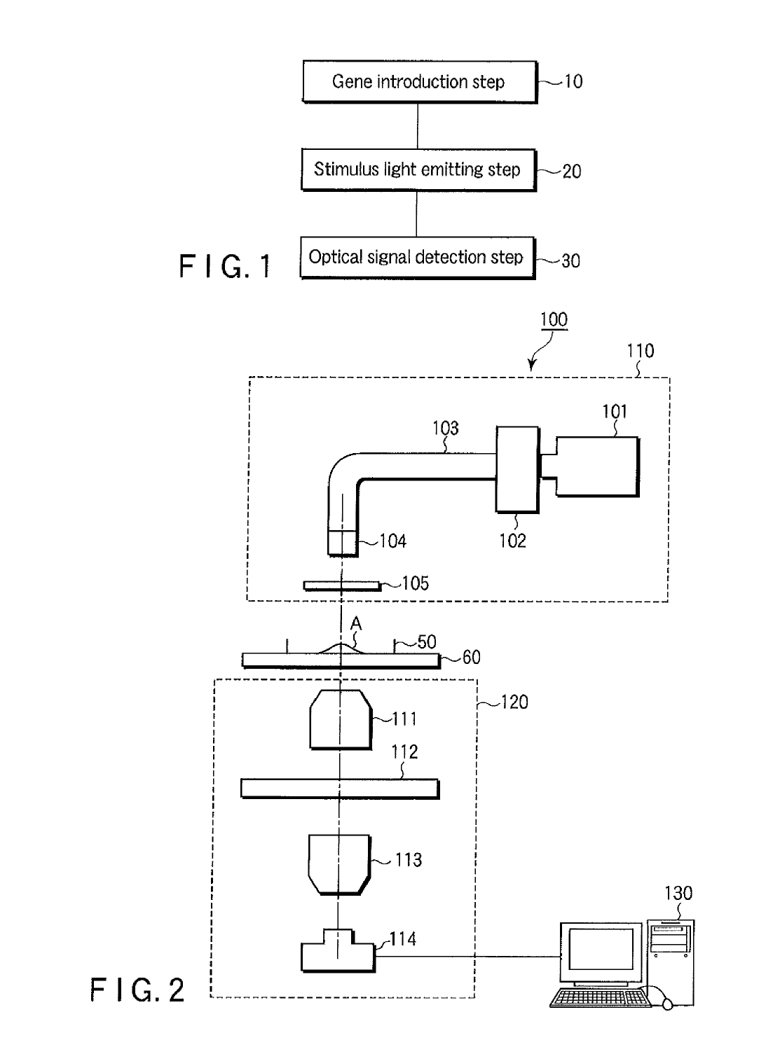

FIG. 1

FIG. 1 is a flow chart of a method of analyzing an optical signal in the first embodiment.

The first embodiment is, as an assumption, a method of analyzing an optical signal which analyzes a signal protein induced by a photosensitive protein (hereinafter, also called a target signal protein).

The photosensitive protein used in the first embodiment means various kinds of proteins activated by photic stimulation of a specific wavelength. Examples of the photosensitive protein include a photosensitive channel protein in which depolarization and hyperpolarization of a channel (that is, opening/closing of a channel) are caused by photic stimulation of a specific wavelength and include, but are not limited to, a photosensitive ion channel film protein derived from green algae such as channel rhodopsin-2 (ChR2) and halorhodopsin (NpHR).

(1) Gene Introduction Step

First, a gene that expresses a luminescent probe to analyze a target signal protein is introduced into an organism sample (10).

The “target signal protein” means a signal protein induced by the photosensitive protein. More specifically, the “target signal protein” is a protein group constituting a series of reaction systems initiated by activation of a specific protein (hereinafter, also referred to as an initiator protein) such as a receptor. A gene that determines a phenotype (that is, a phenotypic determination gene) positioned most downstream of a signal system is expressed in the end after passing through a reaction cascade by the signal protein. In other words, the target signal protein in the first embodiment is an intracellular signal transmitter and means a so-called second messenger. Various kinds of second messengers exist and typical examples thereof include cyclic AMP (cAMP), inositol trisphosphate (IP₃), diacylglycerol (DG), and intracellular free calcium.

The organism sample used in the first embodiment is not particularly limited and, for example, any organism selected from the group consisting of animals (excluding humans), plants, fungi, eukaryotic unicellular organisms, and prokaryotic organisms may be used or any organism species may be selected. The organism sample may also be one of various organs extracted from an individual organism or a tissue fragment thereof, or cells extracted from the tissue fragment. Alternatively, like a vital observation of small animals (such as mice), which is called in vivo imaging, the present embodiment may be applied to a scene in which medical phenomena or drug reactions inside the body of non-human animals are observed by directly accessing living individual animals.

Genes that express a luminescent probe to analyze a target signal protein (hereinafter sometimes referred to as luminescent probe gene) include a gene that expresses a luminescent probe capable of inducing light emission by adding any luminescent substrate. Typical examples thereof include a luciferase gene derived from various animals such as Photinus pyralis and Renilla reniformis. Luciferase oxidizes luciferin, which is a luminescent substrate, to induce light emission. As another example, aequorin, which is a photoprotein derived from Aequorea coerulescens, is known. Aequorin is a complex of apoaequorin, which is a calcium-binding protein, and coelenterazine, which is a luminescent substrate. With calcium bound thereto, a higher-order structure changes and blue light is induced after the substrate is oxidized. Obeline, which is a photoprotein similar to aequorin, is a photoprotein particularly suitable for calcium imaging. Obeline is also a conjugated protein of apoobeline (calcium-binding protein) and coelenterazine (luminescent substrate) and after calcium is bound thereto, blue light near 490 nm is emitted. An apoobeline gene is available from, for example, Lux biotechnology.

The luminescent probe gene can be introduced into an organism sample by any gene recombination technology known to those skilled in the art. For example, the luminescent probe gene may be integrated into an expression vector such as plasmid so that the expression vector can be introduced into an organism sample by using the transfection using DNA-Ca phosphate coprecipitation, particle gun method, electroporation, or microinjection. Alternatively, the luminescent probe gene may be introduced into an organism sample by using infectivity of an adenovirus or retrovirus vector. Further, a transgenic organism that expresses the luminescent probe gene may be produced.

Incidentally, a gene that expresses a photosensitive protein (hereinafter, also called a photosensitive protein gene) may be introduced into the organism sample together with the luminescent probe gene. When the expression of a target signal protein is evaluated by using an organism sample that sufficiently expresses the photosensitive protein, the photosensitive protein gene need not necessarily be introduced. However, when an organism sample that does not at all express, or only slightly expresses the photosensitive protein is used, it is necessary to construct an experiment system suitable for evaluating the expression of a target signal protein. In such a case, a photosensitive protein gene can be introduced into the organism sample together with the luminescent probe gene. By newly introducing the photosensitive protein gene, a start signal for photic stimulation is saturated so that a signal transmission system positioned downstream is sufficiently promoted. As a result, imaging of the signal transmission system can be performed with more sensitivity.

In general, a photosensitive protein, particularly a photosensitive channel protein has a quick response time after photic stimulation so that the downstream signal transmission system operates immediately after the photic stimulation. Thus, if the signal transmission system after photic stimulation should be analyzed, a photic stimulation system and a signal analysis system cannot be constructed as independent systems. Therefore, the analysis system of the signal gene (second messenger) is clearly different an analysis system of various genes that can be constructed as an independent analysis system itself in which a phenotypic determination gene or the like is expressed when quite a long time passes after photic stimulation. The present invention has been made to settle the issue of light interference that has been a problem of the analysis system of the signal gene forced to overlap with a photic stimulation system. Since an analysis system of phenotypic determination gene itself can be constructed as an independent system, an issue of light interference does not arise in the first place.

(2) Stimulus Light Emitting Step

Subsequently, a stimulus light to activate the photosensitive protein is irradiated (20).

The stimulus light is a stimulus suitable for activating the target photosensitive protein and, for example, among the aforementioned photosensitive channel proteins, ChR2, a sodium ion channel, responds to photic stimulation of blue near 470 nm and NpHR, chlorine ion channel, responds to photic stimulation of orange near 580 nm. With the channel protein activated, the downstream intracellular signal transmitter (second messenger) is activated to transmit a series of signals.

(3) Optical Signal Detection Step

Lastly, an optical signal emitted by the organism sample is detected (30).

The optical signal is a luminescent signal by a luminescent probe used to observe the expression of a target signal protein and if, for example, a luciferase gene is introduced, luciferin, which is a luminescent substrate, is oxidized by luciferase generated by the expression of the gene into oxyluciferin, during which yellow light near 530 nm is emitted. Locality of the target signal protein can be identified by detecting generated emission using a pickup unit such as a CCD camera and performing image processing. Moreover, the amount of expression can precisely be quantified for each expression site of the target signal protein by measuring the quantity of optical signal at each position identified by the image processing.

A luminescent probe is used for imaging a target signal protein and thus, it is necessary for the luminescent probe to be expressed in conjunction with the target signal protein. If, for example, luciferase is used as a luminescent probe, the expression of the target signal protein can precisely be imaged by constructing a vector in which a luciferase gene is integrated into a position that allows coexpression with a gene that codes the target signal protein. On the other hand, a luminescent probe like aequorin and obeline emits light by being bound to intracellular free calcium, which is a second messenger, and thus, the expression of calcium can precisely be imaged by adding coelenterazine, a luminescent substrate, to the organism sample in advance.

### Second Embodiment

As the second embodiment, two target signal proteins or more to be analyzed may be present.

When, for example, two target signal proteins (first and second signal proteins) are detected, a gene to express a first luminescent probe (first luminescent probe gene) to analyze the first signal protein and a gene to express a second luminescent probe (second luminescent probe gene) to analyze the second signal protein are introduced into an organism sample. The first and second luminescent probe genes code luminescent probes emitting lights of mutually different wavelengths, and light emitted by each luminescent probe is identified as a different optical signal so that the light can individually be imaged and quantified.

The first and second signal proteins are imaged by the first and second luminescent probes respectively and thus, expressions of each signal protein and each luminescent probe need to be mutually linked.

When, for example, calcium ions (first signal transmitter) and a c-Fos protein (second signal protein) induced by a photosensitive ion channel protein such as ChR2 and NpHR are monitored by using luminescent probes, obeline (first luminescent probe) can be used as the luminescent probe of calcium ions and luciferase (second luminescent probe) as the luminescent probe of the c-Fos protein.

In the above case, the luciferase gene is arranged adjacent to a promoter of a c-fos gene that codes the c-Fos protein. By introducing a vector in which the luciferase gene is arranged adjacent to the promoter of the c-fos gene into a cell (organism sample), both genes are coexpressed in the cell and, as a result, the expression of the c-fos gene can be imaged through luciferase. Incidentally, the c-Fos protein, which is a product of the c-fos gene, is dimerized with c-Jun, an intranuclear protein, to constitute a transcription factor c-Fos/AP-1 complex. c-Fos/AP-1 is bound to a specific AP-1 binding site on the gene promoter to promote expressions of downstream genes.

Luciferin and coelenterazine (hereinafter, also referred to as luciferin or the like), which are luminescent substrates, are introduced into the organism sample. Methods of introducing luciferin or the like include, for example, a method of directly spraying a solution of luciferin or the like to an observation target site and a method of adding luciferin or the like to a solution holding the organism sample such as a culture solution.

When imaging two target signals or more, overlapping of wavelengths of excitation light and stimulus light becomes more intensive according to conventional detection of fluorescence, aggravating the problem of light interference. On the other hand, according to the imaging method of the present invention, a target signal is imaged using a luminescent probe and thus, the problem of light interference does not arise even if the number of target signals increases, making the imaging method a very effective technique.

2. Optical Signal Analysis System
FIG. 2

FIG. 2 is a schematic diagram of an optical signal analysis system 100 used in the first embodiment.

The optical signal analysis system 100 in FIG. 2 is an optical signal analysis system to analyze a signal protein induced by an observational photosensitive protein and having a so-called inverted optical design in which an observation target is observed from below, and comprises a stimulus light emitting unit 110 to irradiate the photosensitive protein with stimulus light to activate the photosensitive protein and a luminescent image pickup unit 120 to pick up an luminescent image in which an optical signal emitted from the organism sample is formed.

As the stimulus light emitting unit 110, any light emitting unit capable of emitting a stimulus light of a suitable wavelength to activate a target photosensitive protein can be used. As a concrete configuration thereof, for example, the stimulus light emitting unit 110 comprises a light source 101, a spectral filter 102, an optical fiber 103, a condensing lens 104, and a shutter 105. Stimulus light emitted from the light source 101 is separated into a plurality of stimulus lights having different wavelength regions through the spectral filter 102 and, among the separated stimulus lights, the stimulus light having a wavelength suitable for activating the target photosensitive protein is irradiated on an organism sample A through the optical fiber 103 and the condensing lens 104. By irradiating cells with, for example, blue light near 470 nm, the photosensitive ion channel protein ChR2 is optically stimulated and sodium ions flow in to depolarize the cells. A calcium transmission system works in the depolarized cell to promote the expression of the c-fos gene. The shutter 105 switches emission of the stimulus light on the organism sample A by transmitting or blocking the stimulus light emitted from the optical fiber 103.

The organism sample A is observed, for example, in a state in which the organism sample A is accommodated in a sample container 50. Examples of the sample container 50 include, but are not limited to, a petri dish, slide glass, microplate, gel support, particulate carrier, and porous filter and may be any accommodation unit made of material such as optically transparent glass, plastics, and resin. The sample container 50 is arranged on an observation stage 60 having an opening or observation window provided at the bottom thereof to allow observation from below.

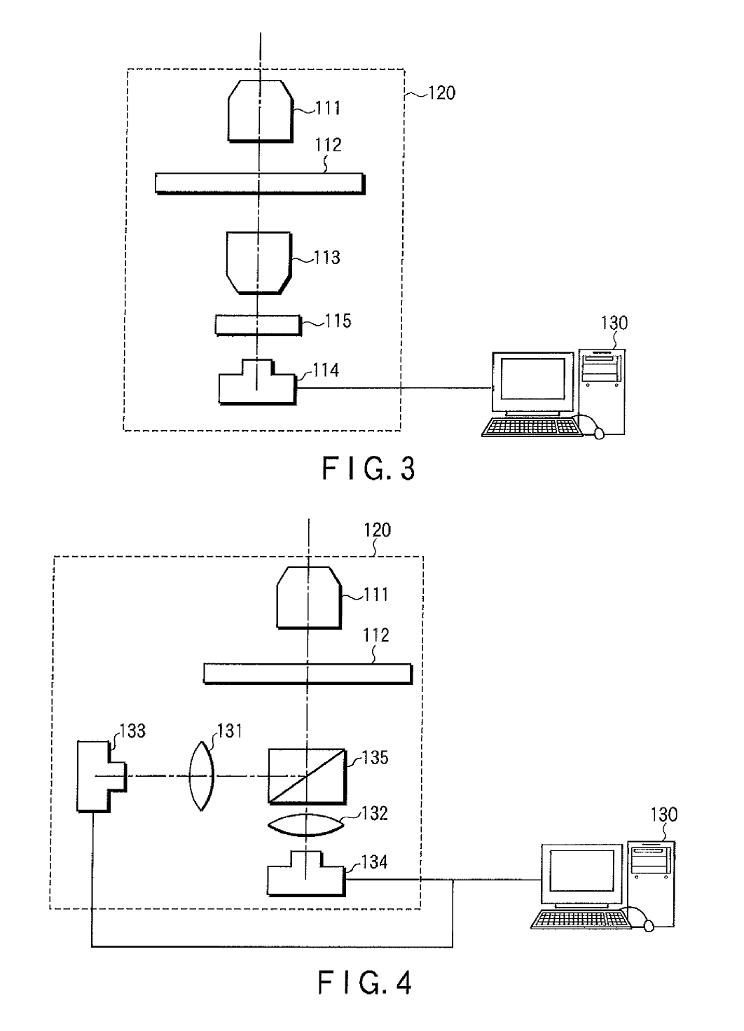

The luminescent image pickup unit 120 may be any pickup unit capable of picking up a luminescent image in which an optical signal emitted from the organism sample is formed. As a concrete configuration thereof, for example, the luminescent image pickup unit 120 comprises an objective lens 111, a luminescent spectral filter 112, an image formation lens 113, and a CCD camera 114. Light emitted from the organism sample A passes through the objective lens 111 to reach the luminescent spectral filter 112. The optical condition that “the value of (numerical aperture/magnification)² is 0.01 or more” is preferably satisfied by the objective lens 111 and/or the image formation lens 113. The luminescent spectral filter 112 as a stimulus light blocking unit blocks the stimulus light used for photic stimulation of the organism sample A and allows only light emitted from the organism sample A to pass toward the camera (for example, a CCD camera or CMOS camera) as a detection unit. After passing through the luminescent spectral filter 112, the light passes through the image formation lens 113 before being detected by the CCD camera 114. A luminescent signal detected by the CCD camera is sent to a personal computer 130 and image processing and light quantity measurement are performed using various kinds of publicly known software to analyze behavioral characteristics of the target signal protein in conjunction with the luminescent signal.
FIG. 3

FIG. 3 is a first schematic diagram of the luminescent image pickup unit 120 in the optical signal analysis system used in the second embodiment.

The basic configuration of the optical signal analysis system is the same as that in FIG. 2, but includes the luminescent image pickup unit 120 capable of detecting two luminescent probes or more separately. As the luminescent image pickup unit 120, it is possible to use any pickup unit capable of picking up a luminescent image in which, among optical signals emitted from an organism sample, an optical signal derived from the first signal protein and that derived from the second signal protein are separately formed. As a concrete configuration thereof, for example, in addition to the configuration shown in FIG. 2, a band-pass filter 115 may be installed between the image formation lens 113 and the CCD camera 114 to detect light emitted from the organism sample by wavelength. When, for example, light emission by obeline or luciferase is detected, the band-pass filter 115 near 490 nm may be arranged on an optical path when light emission by obeline is received. On the other hand, when light emission by luciferase is received, the band-pass filter 115 near 530 nm may be arranged on the optical path. Switching of the band-pass filter may be manual or automatic.
FIG. 4

FIG. 4 is a second schematic diagram of the luminescent image pickup unit 120 in the optical signal analysis system used in the second embodiment.

Instead of providing the band-pass filter 115, a dichroic mirror 135 may be installed on the optical path to separate light emission obtained from the organism sample by wavelength. Image formation lenses 131 and 132 and CCD cameras 133 and 134 are installed respectively in each of branched optical paths ahead to be able to detect light emissions of different wavelengths separately. According to the configuration in FIG. 4, switching of the band-pass filter is not needed and optical signals can be detected at the same time. Thus, two target signal proteins can be imaged simultaneously and continuously. This is particularly useful, for example, when two target signal proteins or more positioned at the same step in a signal transmission system are detected.

In the above embodiment, the stimulus light blocking unit selectively blocks light in accordance with the type such as the wavelength and allows other light to pass and thus, photic stimulation and luminescent signal detection can be carried out simultaneously. Thus, if a signal analysis including the instant of photic stimulation is carried out or photic stimulation is provided in any timing and/or stimulation time (pulse-formed or continuous stimulation), a correct analysis can always be carried out without missing a detection light obtained immediately after the photic stimulation. While the above embodiments adopt the inverted optical design in which observation is made from below, an erecting design in which an observation target is observed from other directions, for example, from above may be adopted or an optical device of a type that accesses an observation target from any direction such as an endoscope may be used.

In the above description, among visual cells necessary for vision, rhodopsin, which is photoreceptive pigment in rod cells, has been described, but the present invention can also be applied to photopsin in cone cells. Moreover, other than such a receptor, the present invention is considered to be also applicable to ganglion cells (ipRGC), which are known to have a projection pathway to the suprachiasmatic nucleus. By applying the present invention to these various photoreceptors, an examination on sensitivity about melatonin secretion or a contribution to a phototherapy that improves secretion can be expected. It is effective to carry out analyses by changing the illuminance of light and color temperature as optical parameters related to the phototherapy in various ways. For example, by determining the relationship between the secretion quantity of melatonin and the circadian rhythm based on action of strong illuminance (example: 1500 to 5000 1×) or weak illuminance (example: 100 to 500 1×) by short wavelength light of 500 nm or less (particularly near 484 nm) at night regarding the illuminance of light or action of light with a high color temperature (example: 3500 to 5000 K) or a low color temperature (example: 1000 to 2700 K) regarding the color temperature, a contribution to improving various morbid states related to dysrhythmia can be made. The circadian rhythm is related also to a chronotherapy and thus, a contribution to improving reactions of medication such as an anticancer agent and antiallergic drug may be made.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

Explanations of Reference Numerals
10: Gene introduction step, 20: Stimulus light emitting step, 30: Optical signal detection step, 50: Sample container, 60: Observation stage, 100: Optical signal analysis system, 101: Light source, 102: Spectral filter for excitation, 103: Optical fiber, 104: Condensing lens, 105: Shutter, 110: Stimulus light emitting unit, 111: Objective lens, 112: Luminescent spectral filter, 113: Image formation lens, 114: CCD camera, 115: Band-pass filter, 120: Luminescent image pickup unit, 130: Personal computer, 131/132: Image formation lens, 133/134: CCD camera, 135: Dichroic minor, A: Organism sample

1-10. (canceled)

11. A method of analyzing an optical signal which analyzes first and second signal substances induced by a photosensitive protein, comprising the steps of:
introducing a gene which expresses a first luminescent probe to analyze the first signal substance and a gene which expresses a second luminescent probe to analyze the second signal substance into an organism sample;
emitting a stimulus light to activate the photosensitive protein; and
detecting, among the optical signals emitted by the organism sample, an optical signal derived from the first signal substance and an optical signal derived from the second signal substance.

12. The method according to claim 11, wherein the organism sample includes a gene which expresses the photosensitive protein.

13. The method according to claim 11, further comprising introducing a gene which expresses the photosensitive protein into the organism sample in the step of introducing a gene.

14. The method according to claim 11, wherein the photosensitive protein is a channel protein.

15. The method according to claim 11, wherein the signal substance is an intracellular signal transmitter.

16. The method according to claim 11, wherein the step of detecting an optical signal is a step of picking up a luminescent image in which, among optical signals emitted by the organism sample, the optical signal derived from the first signal substance and the optical signal derived from the second signal substance are separately formed.
